(19) United States
(12) Patent Application Publication
(10) Pub. No.: US 2015/0073221 A1
(43) Pub. Date: Mar. 12, 2015

(54) DUAL CHANNEL SURGICAL DEVICE FOR ABDOMEN ACCESS

(71) Applicant: Vincenzo NUZZIELLO, (US)

(72) Inventor: Vincenzo NUZZIELLO, Foggia (IT)

(21) Appl. No.: 14/391,396

(22) PCT Filed: Apr. 10, 2013
(86) PCT No.: PCT/IB2013/000649

§ 371 Date: Oct. 9, 2014

(30) Foreign Application Priority Data
Apr. 11, 2012 (IT) BA2012U000020

(51) Int. Cl. A61B 17/34; A61B 17/02

(52) U.S. Cl. CPC A61B 17/3423; A61B 17/0218; A61B 17/3498; A61B 2017/3449; A61B 2017/347
USPC 600/204

(57) ABSTRACT

A trocar for performing laproscopic surgery is disclosed. The trocar is equipped with a double channel allowing the simultaneous introduction of an optical instrument and a grasping or cutting instrument. Furthermore, the introduction of a movable two-way valve allows the rapid emptying of the abdomen or the removal of important anatomical pieces, if necessary.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present patent concerns a medical/surgical device, called trocar, used for laparoscopic surgery.

2. Description of Related Art Including Information Disclosed Under 37 CFR 1.97 and 37 CFR 1.98.

Laparoscopic surgery is a surgical technique in which operations are performed without laparotomy but through the use of a camera connected to a monitor and surgical instruments such as scissors, clamps, electrocoagulators, staplers, needle holders, etc., which are introduced through small incisions into the abdominal wall. To this purpose, a gas, usually carbon dioxide or nitrogen protoxide, is first injected into the abdominal cavity in order to create the necessary space for manipulations of the surgical instruments. Subsequently, a camera is introduced and, under direct visual control, the other trocars are introduced to provide for the insertion of the surgical instruments.

In particular, the trocar according to the present invention consists of two channels, in other words it has two cannulas, parallel to one another and capable of accommodating, at the same time, a camera and a surgical instrument or two surgical instruments.

As known, in order to perform laparoscopic surgery the patient must not have previously undergone surgery using the same technique.

The main obstacle is represented by the presence of postoperative adhesions which sometimes constitute an insurmountable wall. Mastery and constant use of the laparoscopic technique has increasingly expanded the applications range, including the approach to previously operated abdomens.

The main difficulty remains, however, the entry and the limited view in suitable areas to obtain sufficient triangulation to perform any procedure. In these cases it is therefore necessary to open a way amongst the adhesions to create a sufficiently wide space to introduce the trocars.

Another problem is represented by the increasingly frequent use of diagnostic laparoscopy valid and useful especially in the staging of abdominal neoplasms. In these cases, the reduced invasiveness and the better overall vision, along with the better clinical results and a greater appreciation for the procedure by the patient, constitute significant advantages.

### BRIEF SUMMARY OF THE INVENTION

The purpose of this patent is to solve the problems associated with laparoscopic surgery, ensuring better efficiency, greater safety, minimally invasive procedure and maximal utility, responding to the current surgical trend requiring access through one umbilical incision, otherwise called “Single port for two”.

A further purpose is that of equipping the jacket for the insertion of the trocar with a two-way movable valve which not only is an excellent seal of the pneumoperitoneum, but which can be removed, if necessary, in case there is the need to quickly empty the abdomen or to remove important anatomical pieces from this access.

A further purpose is that of providing a trocar through which a camera and a grasping or cutting instrument can be accommodated contemporaneously provided that they do not create an obstacle to each other.

A yet further purpose is that of allowing, through a better placement of taps for the introduction of the C02, the elimination of the edges from the main supporting body allowing, as a consequence, a greater range of motion to the surgeon.

Finally, the trocar aims at reducing as much as possible damage to the patient's internal organs during its introduction into the abdomen, unlike what happens with the introduction of trocars equipped with blade.

### BRIEF DESCRIPTION OF THE DRAWINGS

These and other objectives of the present patent will be illustrated through the following detailed description of the preferred embodiment, which is not intended to limit the scope of the patent, with reference to the attached drawings:

FIG. 1 is an exploded axonometric view of the dual channel surgical device in a preferred embodiment;

FIG. 2 is an axonometric view of the surgical device of FIG. 1 fully assembled;

FIG. 3 is a side view, long side, of the three main elements of the trocar disassembled, of the supporting body;

FIG. 4 is a section view of the trocar;

FIG. 5 is an exploded axonometric view of the single-spindle including the two-way valve and the valve block;

FIG. 6 is a top view of the supporting body;

FIG. 7 is a side view, long side, of the supporting body of FIG. 6;

FIG. 8 is a side view, short side, of the supporting body of FIG. 6 and FIG. 7;

FIG. 9 is a bottom view of the supporting body of FIGS. 6, 7 and 8;

FIG. 10 is an axonometric view of the extractor's seal block;

FIG. 11 is a sectional side view of the extractor;

FIG. 12 is a side view of the extractor.

### DETAILED DESCRIPTION OF THE INVENTION

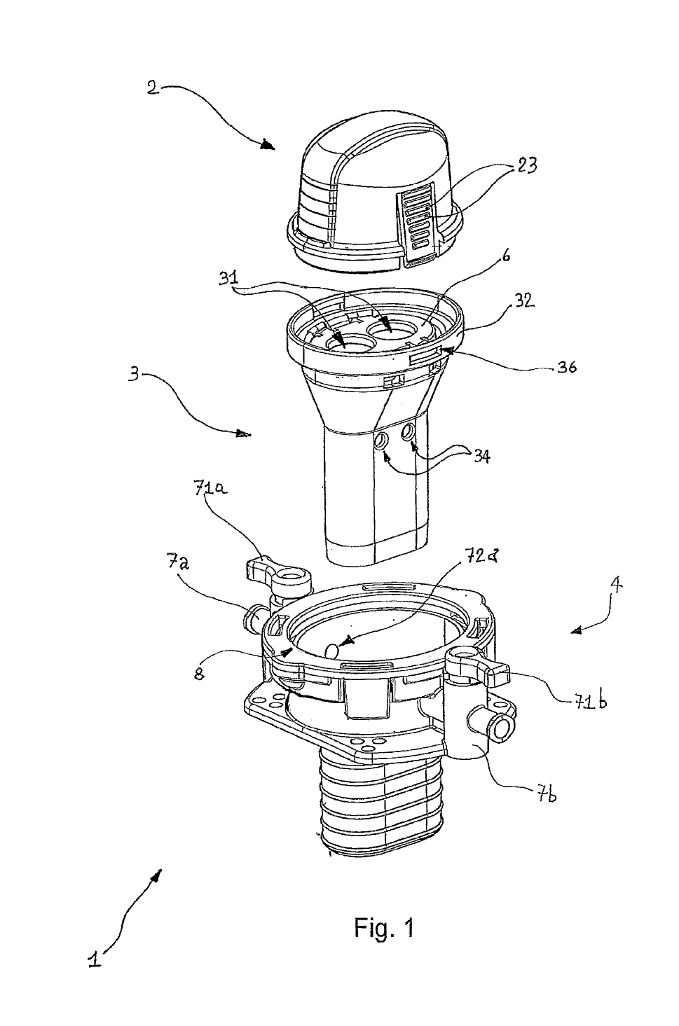

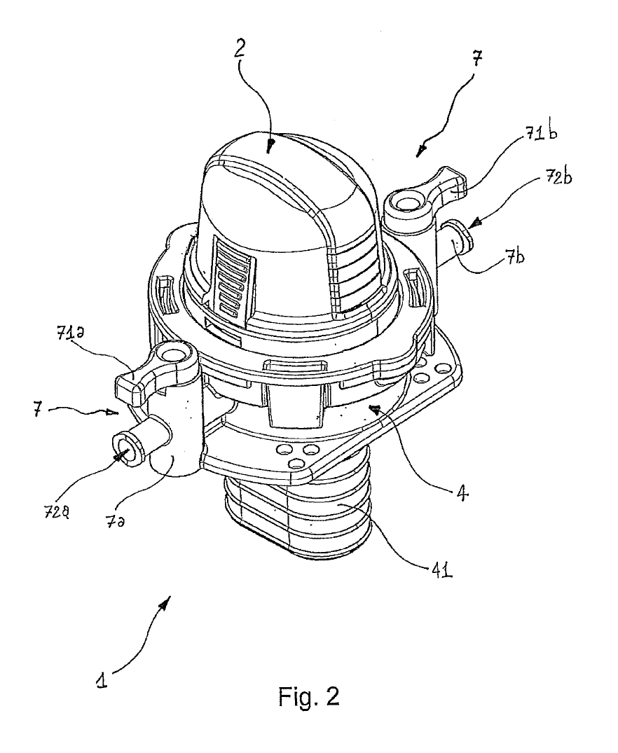

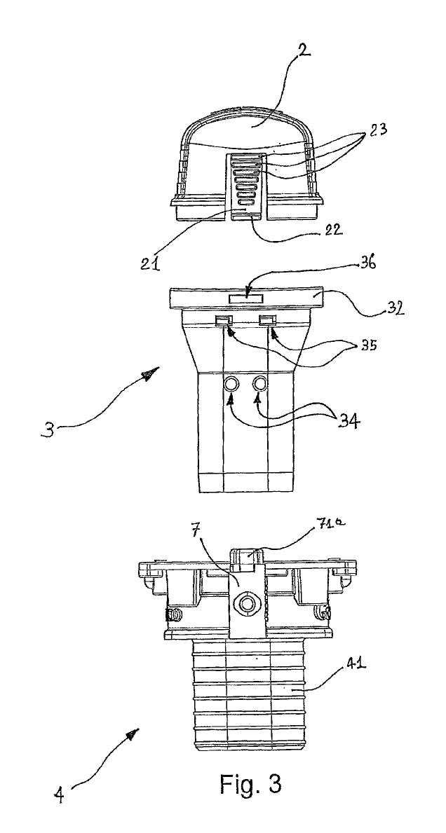

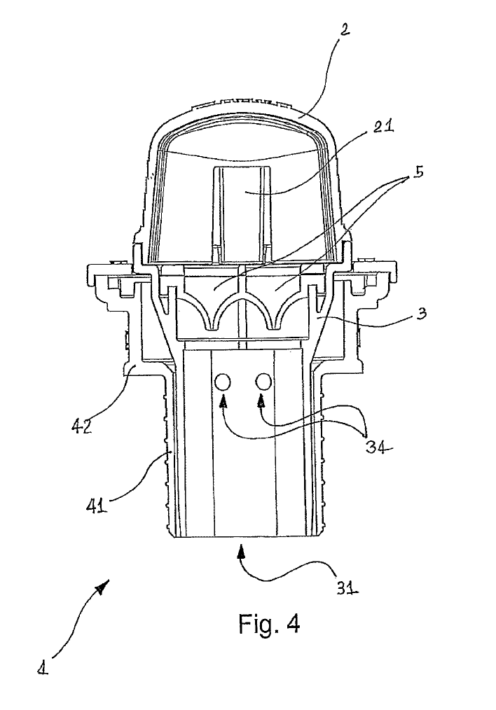

With reference to FIGS. 1, 2 and 3, the surgical device according to the present patent, identified as 1, consists of three main elements: an extractor 2, a single-spindle 3 with two cannulas 31, and a supporting body 4 with incorporated taps 7.

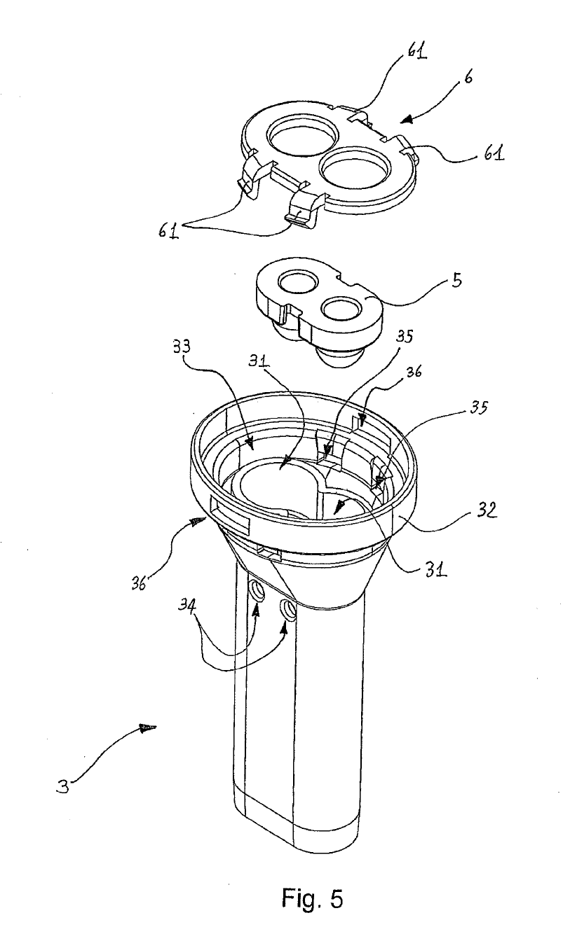

With reference to FIG. 5, the main element of the surgical device is the single-spindle 3 including two cannulas 31 parallel to each other and of equal length and internal diameter, fused together and capable of simultaneously receiving a camera and a grasping or cutting instrument required for the surgery.

The upper opening 33 of the single-spindle 3 has the shape of a funnel 32 and incorporates a silicone rubber two-way movable valve 5 securely fastened to the single-spindle 3 through a valve block 6, equipped, to this purpose, with four fixing teeth 61 fitting in as many seats 35 formed inside the opening 33.

Fastening of the valve block 6 through the aforesaid teeth 61 provides the possibility to remove the two-way movable valve 5, if necessary, to remove important anatomical pieces.

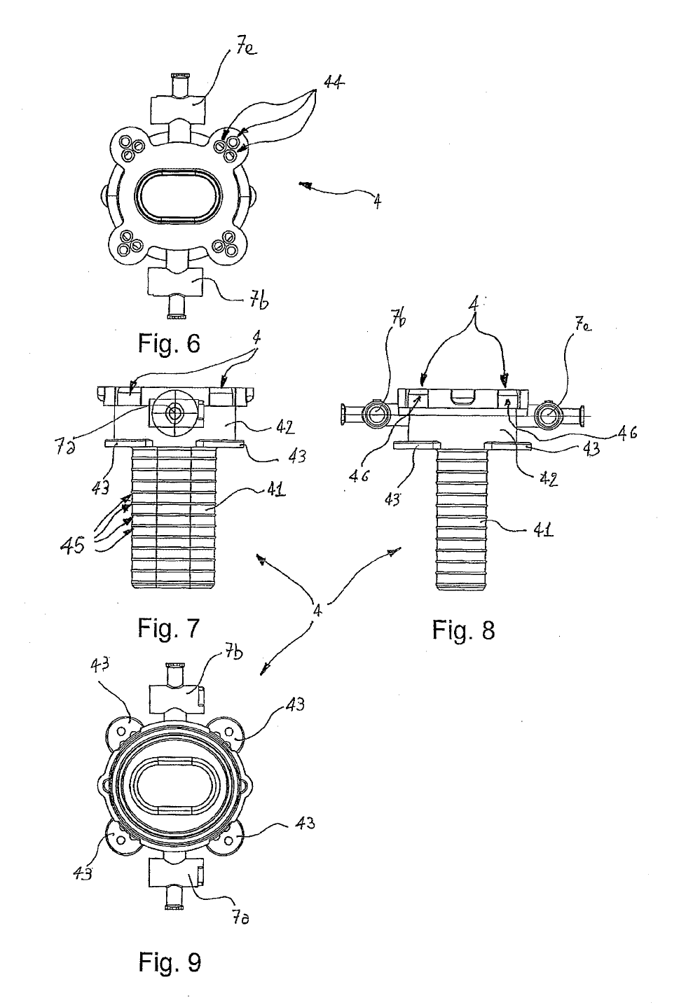

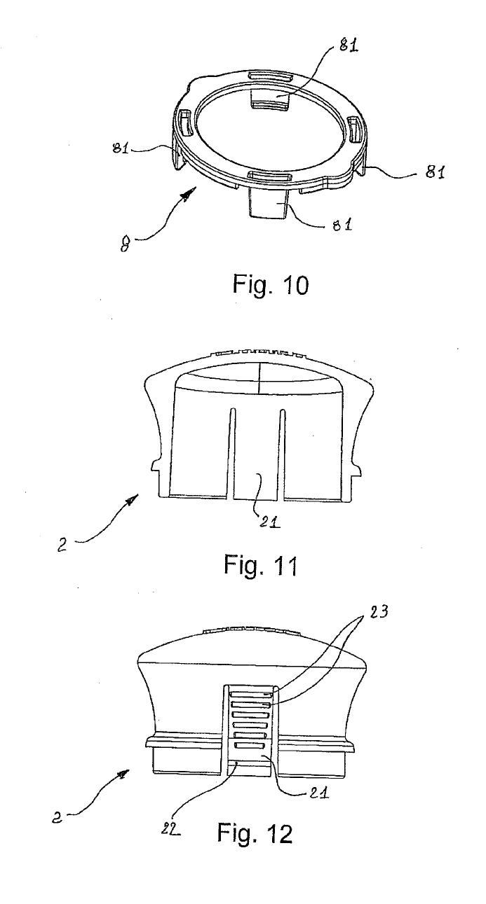

In particular, with reference to FIGS. 6 to 9, a further important element of the surgical device 1 is the body 4 which, together with the single-spindle 3 aims at opening the abdomen, as well as at guiding the insertion of the surgical instruments into the patient's abdomen.

Said body 4 is formed of an oval tube 41, a head 42, two taps 7 incorporated in said head 42, stabilizing fins 43 to stabilize the same body 4 after its introduction into the patient's body. Three holes 44 are provided on said fins 43, to allow the passage of sutures.

Four protrusions 46 are provided outside the head 42 of the body 4 to engage with four elastic teeth 81 perpendicularly positioned to the seal block 8.

The adoption of a fastening by means of the elastic teeth 81 makes the seal block 8 removable from the body 4.

In order to increase the pneumatic seal of the body 4, the outer surface of the oval tube 41 is provided with a series of seal rings 11 which keep the C02 from leaking from the patient's body.

As mentioned, the body 4 is provided with two taps 7 wherein the tap 7a injects and maintains, at the right pressure, the C02 or another gas into the abdominal cavity for the creation of the pneumoperitoneum and the tap 7b for its discharge.

Said taps 7, and in particular the control levers 71, are rotated of 90° with respect to the head of the body 4, making their movement more efficient and natural.

The gas is introduced into the body 4 through a nozzle 72a, its flow is adjusted by the control lever 71a, and it is introduced inside the cannulas 31 through the holes 34. Otherwise, the gas escapes from the body 4 through the nozzle 72b, and its flow is regulated by the control lever 71b.

With reference to FIGS. 11 and 12, the device 1 is equipped with an extractor 2 comprising a pair of elastic elements 21 provided with teeth 22 at their ends. Said teeth are suitable for engaging with the notches 36 formed on the upper edge of the funnel 32 of the single-spindle 3.

In order to allow an easier extraction of the single-spindle 3 from the body 4, the extractor 2 is provided with a knurling 23.

In order to perform laparoscopic surgery, a pneumoperitoneum is first created by the surgeon. This is achieved by inflating the peritoneal cavity with sterile, heated and humidified carbon dioxide to obtain elevation and separation of the abdominal wall from the internal organs. Insufflation may be performed using a Veress needle (with a retractable protection mechanism) which allows the gas necessary to create the pneumoperitoneum to access the peritoneal cavity through a periumbilical incision.

The Veress needle is then extracted and the body 4 is inserted into the same incision, the body 4 being directly connected to the insufflating device through the tap 7a in order to ensure sufficient Insufflation to compensate loss of gas during introduction of the instruments. The same procedure can be performed directly making a cut into the site and entering the body 4 without the aid of the Veress needle, thereby introducing the C02.

Subsequently, after insertion of the single-spindle 3 inside the body 4, the abdominal cavity is reached. It is now possible to introduce the surgical instruments such as the camera to display the cavity and the other surgical instruments.

The main indication of the device is adhesiolysis in previously operated abdomens with laparoscopic technique or with traditional surgery, allowing exploration and removal of the adhesions through a single incision.

Further therapeutic indications are diagnostic and bioptic exploration, staging, cholecystectomy, appendicectomy, hepatic access, ovarian cysts draining out, ovariectomy etc.

1. Dual channel surgical device for abdomen access comprising an extractor (2), a single-spindle (3) and a supporting body (4), characterized in that the single-spindle (3) includes two cannulas (31) of equal length and diameter, fused together into a single piece and placed within said single-spindle (3), said single-spindle (3) being of a size such as to enable its insertion inside said supporting body (4).

2. Surgical device according to claim 1 characterised in that the single-spindle (3) can be removed from the supporting body (4) through an extractor (2), said extractor (2) being equipped, to this purpose, of elastic elements (21) having teeth (22) at their terminal ends, said teeth (22) being suitable to engage with the single-spindle (3) through notches (36) at the upper edge of the single-spindle's funnel (32).

3. Surgical device according to one or more of the preceding claims, characterised in that the extractor (2) is provided with a knurling (23), which is a non-slip surface.

4. Surgical device according to one or more of the preceding claims, characterized by a supporting body (4) formed of an oval tube (41), a head (42), two taps (7) incorporated in said head (42), and stabilizing fins (43).

5. Surgical device according to claims 1 and 4, characterized in that said stabilizing fins (43) are transverse to the head (42) of the body (4), on which body (4) one or more holes (44) are formed.

6. Surgical device according to claims 1, 4 and 5, characterized in that on two opposite sides of the head (42) two taps (7) are placed, wherein the control levers (71) of said taps (7) are rotated of 90° with respect to the head (42) of said body (4).

7. Surgical device according to claims 1, 4, 5 and 6, characterized in that on the outer surface of the oval tube (41) a series of seal rings (45) are placed.

8. Surgical device according to one or more of the preceding claims, characterized in that within the upper funnel-shaped opening (33) of the single-spindle (3) a movable silicone rubber two-way valve (5) is placed, said valve (5) being blocked, when needed, by a valve block (6), said valve block (6) being equipped with four teeth (61) to elastically engage with seats (35) formed inside the opening (33).
